(19) United States
(12) Patent Application Publication
(10) Pub. No.: US 2022/0246298 A1
(43) Pub. Date: Aug. 4, 2022

(54) MODULAR ARCHITECTURE FOR A MEDICAL DIAGNOSTICS DEVICE WITH INTEGRATED ARTIFICIAL INTELLIGENCE CAPABILITIES

(71) Applicant: AI Optics Inc., New York, NY (US)

(72) Inventors: Sayed Mazdak Abulnaga, Somerville, MA (US); Benjamin JJ Villard, Bethesda, MD (US); Andrew DiGiore, New York, NY (US); Luke Michael Moretti, New York, NY (US)

(21) Appl. No.: 17/585,899

(22) Filed: Jan. 27, 2022

Related U.S. Application Data
(60) Provisional application No. 63/144,413, filed on Feb. 1, 2021.

(51) Int. Cl. G16H 50/20; G16H 30/20; A61B 3/12; A61B 3/14; A61B 3/00

(52) U.S. Cl. CPC G16H 50/20; G16H 30/20; A61B 3/0033; A61B 3/14; A61B 3/12

(57) ABSTRACT

A retinal diagnostics instrument can include an imaging device configured to capture image data of an eye of a patient and an electronic processing circuitry configured to execute a communication control method. The communication control method can include providing input data to and receiving output data from a plurality of modules configured to facilitate diagnosing a retina of the eye of the patient using the image data. The communication control method can further includes causing independent processing of input data by any first module of the plurality of modules and any second module of the plurality of modules, any second module being different than any first module, where processing of input data by any first module does not depend on directly providing data to or receiving data from any second module and vice versa.

### CROSS-REFERENCE TO RELATED APPLICATIONS

This application claims priority to U.S. Provisional Application No. 63/144,413 filed on Feb. 1, 2021, which is incorporated by reference in its entirety.

### TECHNICAL FIELD

Disclosed are systems and methods that can perform retinal analysis with medical diagnostics devices, for example, using artificial intelligence (AI) having a modular architecture.

### BACKGROUND

A fundus (or retina) camera is an instrument for inspecting the retina of the eye. Many ophthalmologic, neurologic, and systemic diseases can cause structural abnormalities in the retina, which alter the visual appearance of the retina. These structural and visible abnormalities are known as biomarkers, and they may indicate the presence of a disease. For example, diabetics have high levels of circulating blood sugar that, over time, can cause damage to the small vessels in the retina and lead to the formation of microaneurysms. Such microaneurysms indicate the presence of diabetic retinopathy, which is a diabetes complication that affects eyes, caused by damage to the blood vessels of the light-sensitive tissue at the retina. Clinicians use fundus cameras to visualize and assess a patient's retina for biomarkers in order to diagnose the disease.

### SUMMARY

In some implementations, a retinal diagnostics instrument can include a housing and an imaging device, which can be supported by the housing. The imaging device can be configured to capture image or video data of an eye of a patient. The retinal diagnostics instrument can include an electronic processing circuitry, which can be supported by the housing. The electronic processing circuitry may include a memory and one or more processors. The memory may store executable instructions that, when executed by the one or more processors, cause the one or more processors to execute a communication control method. The communication control method may include providing input data to and receiving output data from a plurality of modules stored in the memory. The plurality of modules can be configured to facilitate diagnosing a retina of the eye of the patient using the image data (which may be obtained from the video data). The communication control method may include causing independent processing of input data by any first module of the plurality of modules and any second module of the plurality of modules Any second module can be different than any first module. Processing of input data by any first module may not depend on directly providing data to or receiving data from any second module and vice versa.

The diagnostics instrument of the preceding paragraph or any of the diagnostics instruments disclosed herein can include one or more of the following features. The communication control method can include providing input data to and receiving output data from the plurality of modules over a plurality of channels. A module of the plurality of modules can be configured to join a channel of the plurality of channels to receive input data or provide output data. The communication of data over the plurality of channels may be performed using one or more key and value pairs. The one or more key and value pairs may be specific to each channel of the plurality of channels. The communication control method can include determining status of the plurality of modules by transmitting requests and receiving status information from the plurality of modules over a plurality of watchdog channels.

The diagnostics instrument of any of the preceding paragraphs or any of the diagnostics instruments disclosed herein can include one or more of the following features. The executable instructions can cause deactivating or upgrading any module of the plurality of modules or adding one or more new modules. Deactivating, upgrading, or adding may not affect operation of any of the other modules of the plurality of modules. At least one of any first module or any second module can include a machine learning module configured to identify one or more diseases of the retina. The imaging device may include a camera. The instrument can include a cup positioned at a distal end of the housing. The cup can be configured to be an interface between instrument and the eye of the patient. The cup may be disposable. The housing may be portable. The housing may include a body and a handle connected to the body and configured to be held by a user. The body may include a user interface, which can be one or more of a display (such as, a touch screen display) or a number of buttons to interface with the user. Any first or any second (or any third module) may receive the inputs from a user interface user and pass inputs to one or more of the other modules. The inputs can include one or more of patient information, selection of a portion of the image data, or quality assessment of the image data.

A method of operating the instrument of any of the preceding paragraphs or any of the instruments disclosed herein is provided.

### BRIEF DESCRIPTION OF DRAWINGS

Throughout the drawings, reference numbers may be re-used to indicate correspondence between referenced elements. The drawings are provided to illustrate example embodiments described herein and are not intended to limit the scope of the disclosure.

FIG. 1 illustrates a retina camera.

FIG. 2 schematically illustrates a system level diagram showing retina camera components of FIG. 1.

FIG. 3 illustrates a flow chart of a process for modular image analysis.

FIGS. 4-7 schematically illustrate modular architectures for a medical diagnostics device.

### DETAILED DESCRIPTION

### Introduction

Current trend is to implement AI systems in the cloud, which provides a framework with nearly infinite processing power. Processing in the cloud can lead to security and privacy issues, and may prevent the use in settings without a fast internet connection, such as, in resource-constrained settings, rural areas, or underserved communities. To address these issues, it may be desirable to execute AI systems on portable hardware, which has limited memory and processing resources. In many cases, AI systems may require memory and processing resources that are beyond the scope of that can be provided by portable hardware.

AI systems may include multiple models, each focusing on a specific task (such as, to detect multiple features, diseases, or conditions). Due to the limitations of portable hardware, these models may need to be run in series, slowing down the workflow. Also, when models are dependent on each other, a failure in one model can affect the entire workflow.

AI systems may be continually modified by upgrading existing models and adding additional ones (such as, to detect additional features, diseases, or conditions), which may require additional capacity and flexibility. Application demands may be constantly changing, and it is often difficult to modify systems to match demand.

As portable hardware advances at a rapid rate, it is beneficial for systems to be malleable and be run on multiple hardware devices. However, as the number of models in a system increases, communication and data storage can become challenging. As systems grow in complexity, designing additional components can become challenging as it requires considering all existing components and proper ways to communicate and manage inputs and outputs.

Disclosed systems and methods generally relate to a modular architecture for AI workflow and processing to allow for increased functionality when deployed on a portable hardware system. Non-limiting advantages of the disclosed systems and methods include offering complex AI solutions on a portable hardware system with limited processing resources and memory space, allowing for the expansion of disease detection strategy on such hardware, and increasing security due to the closed loop properties (such as, not relying on cloud processing).

Medical Diagnostics Devices with On-Board AI

A device with integrated artificial intelligence (AI) can be used to assess a patient's body part to detect a disease. The device may be portable or handheld by a user (which may be a patient or healthcare provider). For example, the device can be a retina camera configured to assess a patient's eye (or retina) and, by using an on-board AI retinal disease detection system, provide real-time analysis and diagnosis of disease that caused changes to the patient's retina. Easy and comfortable visualization of the patient's retina can be facilitated using such retina camera, which can be placed over the patient's eye, display the retina image on a high-resolution display, potentially with screenshot capabilities, analyze a captured image by the on-board AI system, and provide determination of presence of a disease.

Such retina camera can perform data collection, processing, and diagnostics tasks on-board without the need to connect to another computing device or to cloud computing services. This approach can avoid potential interruptions of the clinical workflow when using cloud-based solutions, which involve transfer of data over the network and, accordingly, rely on network connectivity. This approach can facilitate faster processing because the device can continually acquire and process images without needing intermediary upload/download steps, which may be slow. Such retina camera can potentially improve accuracy (for instance, as compared to retina cameras that rely on a human to perform analysis), facilitate usability (for example, because no connectivity is used to transfer data for analysis or transfer results of the analysis), provide diagnostic results in real-time, facilitate security and guard patient privacy (for example, because data is not transferred to another computing device), or the like. Such retina camera can be used in many settings, including places where network connectivity is unreliable or lacking.

Such retina camera can allow for better data capture and analysis, facilitate improvement of diagnostic sensitivity and specificity, and improve disease diagnosis in patients. Existing fundus cameras may lack one or more of portability, display, on-board AI capabilities, etc. or require one or more of network connectivity for sharing data, another device (such as, mobile phone or computing device) to view collected data, rigorous training of the user, etc. In contrast, allowing for high-quality retinal viewing and image capturing with faster analysis and detection of the presence of disease via on-board AI system and image-sharing capabilities, the retina cameras described herein can potentially provide improved functionality, utility, and security. Such retina camera can be used in hospitals, clinics, and/or at home. The retina cameras or other instruments described herein, however, need not include each of the features and advantages recited herein but may possibly include any individual one of these features and advantages or may alternatively include any combination thereof.

As another example, the device can be an otoscope configured to assess a patient's ear and, by using an on-board artificial intelligence (AI) ear disease detection system, possibly provide immediate analysis and/or diagnosis of diseases of the patient's ear. Such an otoscope can have one or more advantages described above or elsewhere in this disclosure. As yet another example, the device can be a dermatology scope configured to assess a patient's skin and, by using an on-board artificial intelligence (AI) skin disease detection system, possibly provide immediate analysis and/or diagnosis of diseases of the patient's skin. Such a dermatology scope can have one or more advantages described above or elsewhere in this disclosure.

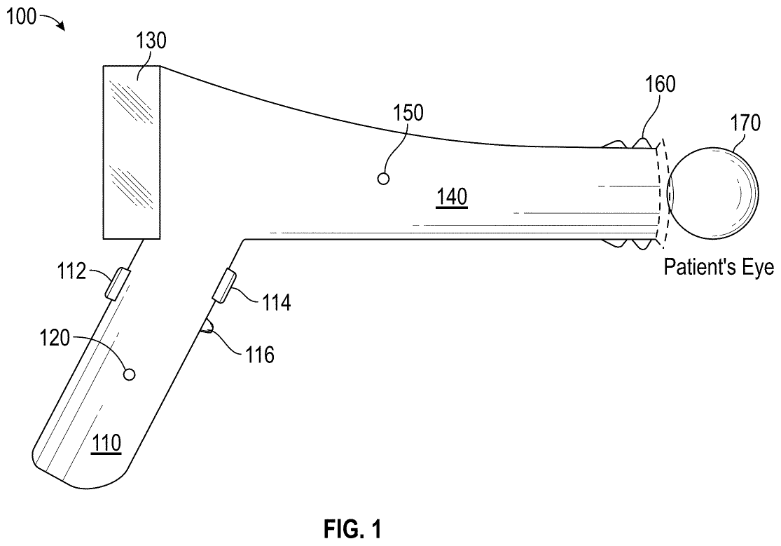

FIG. 1 illustrates an example retina camera 100. A housing of the retina camera 100 can include a handle 110 and a body 140 (in some cases, the body can be barrel-shaped). The handle 110 can optionally support one or more of power source, imaging optics, or electronics 120. The handle 110 can also possibly support one or more user inputs, such as a toggle control 112, a camera control 114, an optics control 116, or the like. Toggle control 112 may be used to facilitate operating a display 130 in case of a malfunction. For example, toggle control 112 can facilitate manual scrolling of the display, switching between portrait or landscape mode, or the like. Toggle control 112 can be a button. Toggle control 112 can be positioned to be accessible by a user's thumb. Camera control 114 can facilitate capturing video or an image. Camera control 114 can be a button. Camera control 114 can be positioned to be accessible by a user's index finger (such as, to simulate action of pulling a trigger) or middle finger. Optics control 116 can facilitate adjusting one or more properties of imaging optics, such as illumination adjustment, aperture adjustment, focus adjustment, zoom, etc. Optics control 116 can be a button or a scroll wheel. For example, optics control 116 can focus the imaging optics. Optics control 116 can be positioned to be accessible by a user's middle finger or index finger.

The retina camera 100 can include the display 130, which can be a liquid crystal display (LCD) or other type of display. The display 130 can be supported by the housing as illustrated in FIG. 1. For example, the display 130 can be positioned at a proximal end of the body 140. The display 130 can be one or more of a color display, high resolution display, or touch screen display. The display 130 can reproduce one or more images of the patient's eye 170. The display 130 can allow the user to control one or more image parameters, such as zoom, focus, or the like. The display 130 (which can be a touch screen display) can allow the user to mark whether a captured image is of sufficient quality, select a region of interest, zoom in on the image, or the like. Any of the display or buttons (such as, controls, scroll wheels, or the like) can be individually or collectively referred to as user interface. The body 140 can support one or more of the power source, imaging optics, imaging sensor, electronics 150 or any combination thereof.

A cup 160 can be positioned on (such as, removably attached to) a distal end of the body 140. The cup 160 can be made at least partially from soft and/or elastic material for contacting patient's eye orbit to facilitate examination of patient's eye 170. For example, the cup can be made of plastic, rubber, rubber-like, or foam material. Accordingly, the cup 160 may be compressible. The cup 160 can also be disposable or reusable. In some cases, the cup 160 can be sterile. The cup 160 can facilitate one or more of patient comfort, proper device placement, blocking ambient light, or the like. Some designs of the cup may also assist in establishing proper viewing distance for examination of the eye and/or pivoting for panning around the retina.

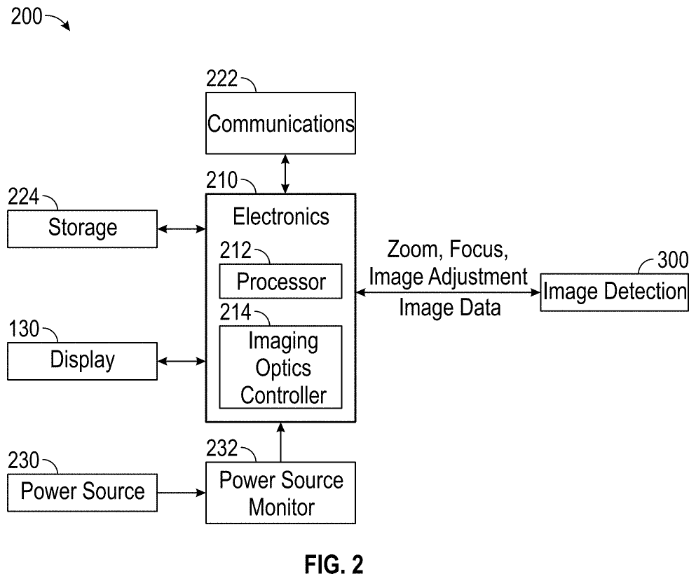

FIG. 2 illustrates a block diagram 200 of various components of the retina camera 100. Power source 230 can be configured to supply power to electronic components of the retina camera 100. Power source 230 can be supported by the handle 110, such as positioned within or attached to the handle 110 or be placed in another position on the retina camera 100. Power source 230 can include one or more batteries (which may be rechargeable). Power source 230 can receive power from a power supply (such as, a USB power supply, AC to DC power converter, or the like). Power source monitor 232 can monitor level of power (such as, one or more of voltage or current) supplied by the power source 230. Power source monitor 232 can be configured to provide one or more indications relating to the state of the power source 230, such as full capacity, low capacity, critical capacity, or the like. One or more indications (or any indications disclosed herein) can be visual, audible, tactile, or the like. Power source monitor 232 can provide one or more indications to electronics 210.

Electronics 210 can be configured to control operation of the retina camera 100. Electronics 210 can include one or more hardware circuit components (such as, one or more controllers or processors 212), which can be positioned on one or more substrates (such as, on a printed circuit board). Electronics 210 can include one or more of at least one graphics processing unit (GPU) or at least one central processing unit (CPU). Electronics 210 can be configured to operate the display 130. Storage 224 can include memory for storing data, such as image data obtained from the patient's eye 170, one or more parameters of AI detection, or the like. Any suitable type of memory can be used, including volatile or non-volatile memory, such as RAM, ROM, magnetic memory, solid-state memory, magnetoresistive random-access memory (MRAM), or the like. Electronics 210 can be configured to store and retrieve data from the storage 224.

Communications system 222 can be configured to facilitate exchange of data with another computing device (which can be local or remote). Communications system 222 can include one or more of antenna, receiver, or transmitter. In some cases, communications system 222 can support one or more wireless communications protocols, such as WiFi, Bluetooth, NFC, cellular, or the like. In some instances, the communications system can support one or more wired communications protocols, such as USB. Electronics 210 can be configured to operate communications system 222. Electronics 210 can support one or more communications protocols (such as, USB) for exchanging data with another computing device.

Electronics 210 can control an image detection system 300, which can be configured to facilitate capturing of (or capture) image data of the patient's eye 170. Electronics 210 can control one or more parameters of the image detection system 300 (for example, zoom, focus, aperture selection, image capture, provide image processing, or the like). Such control can adjust one or more properties of the image of the patient's eye 170. Electronics 210 can include an imaging optics controller 214 configured to control one or parameters of the image detection system 300. Imaging optics controller 214 can control, for example, one or more motor drivers of the image detection system 300 to drive motors (for example, to select an aperture, to select lenses that providing zoom, to move of one or more lenses to provide autofocus, to move a detector array 380 or image sensor to provide manual focus or autofocus, or the like). Control of one or more parameters of the image detection system 300 can be provided by one or more of user inputs (such as a toggle control 112, a camera control 114, an optics control 116, or the like), display 130, etc. Image detection system 300 can provide image data (which can include one or more images) to electronics 210. As disclosed herein, electronics 210 can be supported by the retina camera 100. Electronics 210 may not be configured to be attached to (such as, connected to) another computing device (such as, mobile phone or server) to perform determination of presence of a disease.

### Disease Identification Through Image Analysis

Electronics 210 can include one or more controllers or processors (such as the processor 212), which can be configured to analyze one or more images to identify a disease. For example, electronics 210 can include a processing system (such as, a Jetson Nano processing system manufactured by NVIDIA or a Coral processing system manufactured by Google), a System-on-Chip (SoC), or a Field-Programmable Gate Array (FPGA) to analyze one or more images. One or more images (or photographs) or video can be captured, for example, by the user operating the camera control 114 and stored in the storage 224. One or more prompts can be output on the display 130 to guide the user (such as, “Would you like to capture video or an image?”). Additionally or alternatively, symbols and graphics may be output on the display 130 to guide the user. Image quality can be verified before or after processing the one or more images or storing the one or more images in the storage 224. If any of the one or more images is determined to be of poor quality (for instance, as compared to a quality threshold), the image may not be processed or stored, the user can be notified, or the like. Image quality can be determined based on one or more of brightness, sharpness, contrast, color accuracy, distortion, noise, dynamic range, tone reproduction, or the like. Image quality verification can be done automatically (such as, with one or more machine learning models) or manually by the user input.

One or more preset modes can facilitate easy and efficient capture of multiple images or video. Such one or more preset modes can automatically focus, capture, verify image quality, and store the video or image(s). For some designs the one or more preset modes can switch one or more settings (such as, switch the light source to infrared light), and repeat this cycle without user intervention. In some designs, for example, a preset mode can facilitate obtaining multiple images for subsequent analysis. Such multiple images, for example, can be taken from different angles, use different light sources, or the like. This feature can facilitate automatically collecting an image set for the patient.

The user can select a region of an image for analysis, for instance, by outlining the region on the touch screen display 130, zooming in on region of interest on the display 130, or the like. In some cases, by default the entire image may be analyzed.

One or more machine learning models (sometimes referred to as AI models) can be used to analyze one or more images or video. One or more machine learning models can be trained using training data that includes images or video of subjects having various diseases of interest, such as retina disease (retinopathy, macular degeneration, macular hole, retinal tear, retinal detachment, or the like), ocular disease (cataracts or the like), systemic disease (diabetes, hypertension, or the like), Alzheimer's disease, etc. For example, any of the machine learning models can include a convolution neural network (CNN), decision tree, support vector machine (SVM), regressions, random forest, or the like. One or more machine learning models processing such images or videos can be used for tasks such as classification, prediction, regression, clustering, reinforcement learning, dimensionality reduction. Training of one or more models can be performed using many annotated images or video (such as, thousands of images or videos, tens of thousands of images or videos, hundreds of thousands of images or videos, or the like). Training of one or more models may be performed external to the retina camera 100. Parameters of trained one or more machine learning models (such as, model weights) can be transferred to the retina camera, for example, via retina camera's wireless or wired interface (such as, USB interface). Parameters of one or more models can be stored in the storage 224 (or in another memory of electronics 210). Output of the analysis (sometimes referred to as a diagnostic report) can include one or more of determination of the presence of disease(s), severity of disease(s), character of disease(s), clinical recommendation(s) based on the likelihood of presence or absence of disease(s). A diagnostic report can be displayed on the display 130. The diagnostic report can be stored in electronic medical record (EMR) format, such as EPIC EMR, or other document format (for example, PDF). The diagnostic report can be transmitted to a computing device. In some cases, the diagnostic report but not image data can be transmitted to the computing device, which can facilitate compliance with applicable medical records regulations (such as, HIPPA, GDPR, or the like).

One or more machine learning models can determine the presence of a disease based on the output of one or more models satisfying a threshold. As described herein, images or videos can be analyzed by one or more machine learning models one at a time or in groups to determine presence of the disease. For instance, the threshold can be 90%. When images are analyzed one at a time, determination of presence of the disease can be made in response to output of one or more models satisfying 90%. When images are analyzed in a group, determination of presence of the disease can be made in response to combined outputs of one or more models analyzing the group of images satisfying 90%.

The user can provide information (or one or more tags) to increase accuracy of the analysis by one or more machine learning models. For example, the user can identify any relevant conditions, symptoms, or the like that the patient (and/or one or more patient's family members) has been diagnosed with or has experienced. Relevant conditions can include systemic disease, retinal disease, ocular disease, or the like. Relevant symptoms can include blurry vision, vision loss, headache, or the like. Symptom timing, severity, or the like can be included in the identification. The user can provide patient demographic information (such as, age, gender, and weight). The user can provide such information using one or more user interface components on the display 130, such as a drop-down list or menu. If an electronic medical (or electronic health) record module is present (as described below), the user (or system) can choose for the module to provide patient demographic or other relevant patient information to one or more machine learning models. The user can also provide such information by connecting the device to a communications network or an external device (such as, using the USB interface) and downloading the information from a secured database. One or more tags can be stored along with one or more pertinent images in the storage 224. One or more tags can be used during analysis by one or more machine learning models during analysis and evaluation. One or more images along with one or more tags can be used as training data.

In some cases, the diagnostic report may alternatively or additionally provide information indicating increased risk of disease or condition for a physician's (such as, ophthalmologist's) consideration or indicating the presence (or absence) of disease of condition. Physician can use this information during subsequent evaluation of the patient. For example, the physician can perform further testing to determine if one or more diseases are present.

Image or video analysis, including the application of one or more machine learning models to one or more images or video, can be performed by execution of program instructions by a processor and/or by a specialized integrated circuit that implements the machine learning model in hardware.

Disclosed devices and methods can, among other things, make the process of retinal assessment comfortable, easy, efficient, and accurate. Disclosed devices and methods can be used in physician offices, clinics, emergency departments, hospitals, in telemedicine setting, or elsewhere. Unnecessary visits to a specialist healthcare provider (such as, ophthalmologist) can be avoided, and more accurate decisions to visit a specialist healthcare provider can be facilitated. In places where technological infrastructure (such as, network connectivity) is lacking, disclosed devices and methods can be used because connectivity is not needed to perform the assessment.

### Modular Architecture

Disclosed systems and methods can allow for multiple modules (such as, AI models and a camera application) to be run independently (such as, in series or in parallel) or simultaneously (such as, in parallel) on a hardware system with limited processing power and memory space. This can be achieved by using a master controller (or master controller module) that manages the communication and inputs/outputs of the modules, thereby allowing the modules to be standalone components. The master controller module can be implemented in software or firmware running on at least one processor of a medical diagnostic device, such as the retina camera illustrated in FIG. 1. The master controller module can effectively serve as the processor between all the respective modules, allowing for seamless communication flow between the modules. Having a master controller can facilitate modularity so that the modules can be easily removed, upgraded, or new ones be added.

Disclosed systems and methods can eliminate bottlenecks in some of the traditional AI workflows. By allowing two or more modules (or processes) to run independently and in parallel, the processes are not dependent on the output of each other. Therefore, if an error occurs in a process or a process produces an unexpected result, the other process(es) can be unaffected. The master controller module can perform a respective action based on the unexpected result or the error.

In some implementations, the separate modules may contain AI models to identify features of or in an image. In some cases, these may be medical images, such as retinal images. The separate modules may identify specific features in a retinal image. For example, there may be one module that identifies macular edema, while another module identifies micro hemorrhages, and yet another module identifies exudates. These modules, separate from one another, are not dependent on the output of one another. As another example, the different modules may identify different diseases or conditions within a retinal image. For example, there may be one module that identifies diabetic retinopathy, while another module identifies glaucoma, yet another module identifies macular degeneration, and yet another module identifies heart attack risk.

In some cases, a retinal diagnostics instrument aimed at detecting retinal pathology could implement several AI-based models, such as an AI model for the detection of macular edema and another AI model for the detection of glaucoma. Both models can be individual modules and can be executed in parallel or sequentially. As the input and outputs of the models can be controlled by the master controller module, if the models were to run in parallel, the controller module would feed the images to both modules independently or simultaneously. If the models were to run sequentially, the controller module would send the images to the first model, receive a result (or an error or unexpected result), then still be able to send the images (and/or results and/or other outputs of the first model) to the second model. In case of any error or unexpected result, the master controller module could still feed the input to the other model(s) without stopping the detection by the system. In this manner, any bottleneck can be removed allowing for a more seamless solution.

Simultaneous or independent processing can be achieved using any one or more of the following approaches. One technique involves designing a modular system with a master controller that handles the inputs and outputs to the AI models via a communication protocol, which can utilize one or more data structures or a database to store the appropriate data and results. This can allow for efficient communication across modules configured to service hardware devices (such as, a camera) and AI models that process data. The modules may communicate via different or independent channels, while messages are passed from each channel to the master controller. The master controller can communicate with the channels to check the status of one more modules (such as, down, running idle, etc.), to send inputs (such as, images), to communicate across hardware and software, to obtain results from the AI models, or the like. Communication may be done by sending messages in the format of key-value pairs (sometimes referred to as key and value pairs). Key-value pairs can be unique for a given channel, allowing AI models (and other modules) to be developed independently.

A given module may have access to multiple channels to distinguish its communication partners. For example, for a disease classification AI model, there may be two channels, “classifier channel” and “classifier results channel.” There could be another channel, such as an EMR channel, that feeds into the disease classification AI model. In this structure, the master controller can send messages to multiple channels, thereby allowing AI models to process data in parallel. Modularity can be achieved by creating new channels for additional AI models, deleting channels for obsolete models, or updating the key-value pairs of messages when modifying existing AI models. Furthermore, channels can be created to check the status of individual AI models (such as, periodically), thereby ensuring that one or more AI models are in a ready state and able to process data as it arrives.

The master controller can allow multiple models to be run in parallel as it can command multiple channels to run at once. Portable hardware systems can be constrained by power consumption, memory space, and processing power. To address these constraints, techniques such as memory swapping and freeing and creating idle states for processes may be used. While hardware constraints may pose a problem, the disclosed architectures with a master controller can allow for efficient parallel processing by treating the models as independent components and effectively managing the input and output.

Software containers may be used to keep a module separated from an operating system or software system. A container can store the module and all required libraries as a discrete instance to be used by a portable hardware system without risking compatibility issues. An individual container can contain several modules.

In some cases, the disclosed modular system may allow for a plug-and-play type of approach to updating a portable retinal diagnostics instrument. This could be done through software updates in an easy manner.

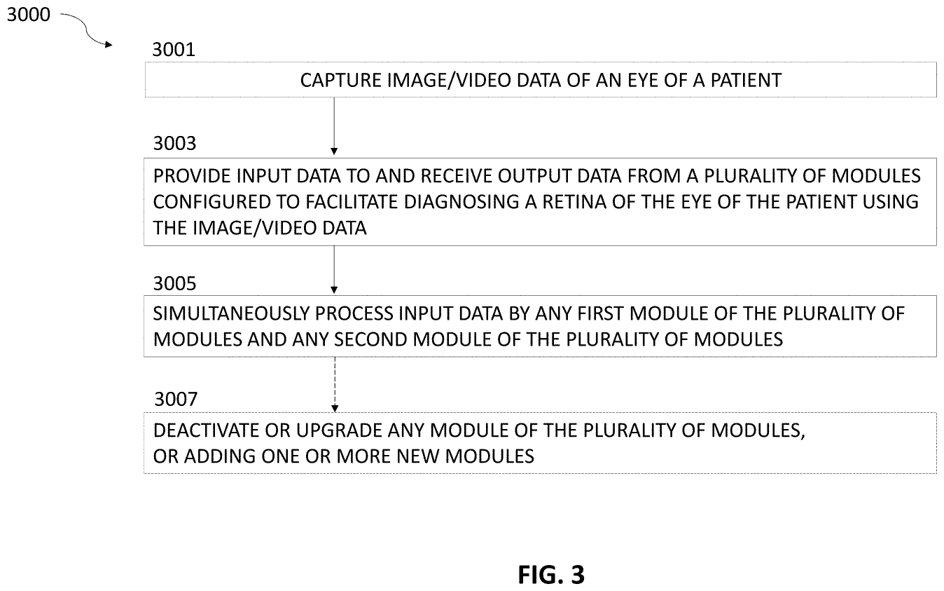

As an example of the disclosed modular AI approach, FIG. 3 illustrates a flow chart of a process 3000 for modular image analysis. The process 3000 can be implemented by a retinal diagnostics instrument, such as the one shown in FIG. 1 and FIG. 2. The retinal diagnostics instrument can include a housing comprising a body and a handle, an imaging device supported by the housing, and an electronic processing circuitry supported by the housing. The handle can be connected to the body and configured to be held by a user. The imaging device (such as, a camera) can be configured to capture image or video data of an eye of a patient. The electronic processing circuitry can include a memory and a processor.

The process 3000 can start at block 3001 where it can capture image or video data of an eye of a patient using the imaging device. After capturing image or video data at block 3001, the process 3000 can move to block 3003 where it can provide input data to and receive output data from a plurality of modules stored in the memory. The plurality of modules can be configured to facilitate diagnosing a retina of the eye of the patient using the image or video data. Image data can be obtained from the video data (for example, images can correspond to video frames), for example, as described in Appendix A. The plurality of modules can be AI models. In some cases, at block 3003, providing input data to and receiving output data from the plurality of modules may be done over a plurality of channels. A module of the plurality of modules can be configured to join (or subscribe to) a channel of the plurality of channels to receive input data or provide output data. In some implementations, communication of data over the plurality of channels is performed using one or more key and value pairs. The one or more key and value pairs can be specific to each channel of the plurality of channels.

Subsequently, the process 3000 can transition to block 3005 where it can cause independent simultaneous processing of input data by any first module of the plurality of modules and any second module of the plurality of modules. Any second module can be different than any first module. Processing of input data by any first module may not depend on directly providing data to or receiving data from any second module and vice versa. In some cases, at least one of any first module or any second module can be a machine learning model configured to identify one or more diseases of the retina. In some implementations, any one of first module or second module can be inputs from the user via the user interface (such, as via a GUI).

Optionally, the process 3000 can transition to block 3007 to cause deactivating or upgrading any module of the plurality of modules, or adding one or more new modules. The deactivating or upgrading or adding may not affect operation of any of the other modules of the plurality of modules.

Blocks 3003, 3005, and 3007 can be executed by a master controller module, which can be executable code stored in the memory of the electronic processing circuitry.

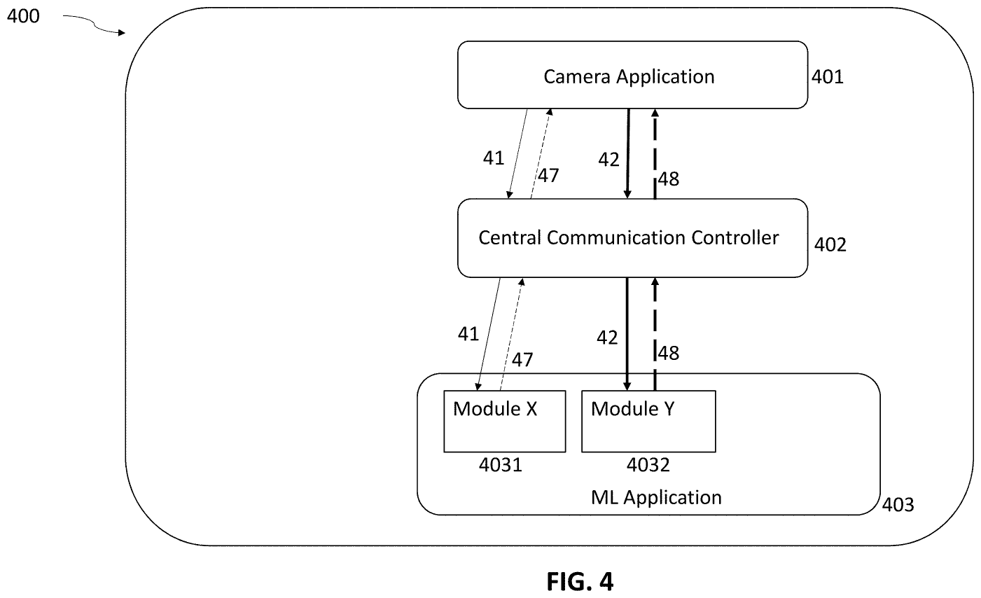

FIG. 4 schematically illustrates a modular architecture 400 for a retinal diagnostics instrument (which can be a closed loop system). The modular architecture 400 can implement the process described in connection with FIG. 3. Any of the modules illustrated in FIG. 4 can be software or firmware modules stored in a memory. The modular architecture 400 can include a camera application module 401, a master controller 402 (sometimes referred to as a central communication controller (CCC)), and a machine learning (ML) application module 403.

The data exchanged between the camera application module 401 and the ML application module 403 may include session data, images, or results. Session data can include patient information or meta data of the images for a session. Session data can be updated as new images are acquired. For example, the camera application module 401 can guide the user to take two images (or video) of the left and right eye. In some cases, a pointer to the images stored in the memory can be part of the session data, but images themselves may not be (in order to minimize the size of the session data). In some implementations, the images can be JPEG files (or another digital image format) and can be stored onto a Linux file system by the camera application module 401. All images for one session may be stored in the same directory. Results produced by the ML application module 403 can be processed by a module (not shown) that causes the data to be displayed to the user (or, in some cases, guide the user to retake the image if the image quality is poor).

The CCC 402 can allow modules to process data independently, regulate their activation or deactivation, upgrade the modules, or the like. For example, in diabetic retinopathy (DR) classification, several modules are needed to go from image capture to classification (which can be expressed as a disease severity score). The ML application module 403 can include two separate modules: an image quality assessment module (Module X, 4031) and a disease classification module (Module Y, 4032). In some cases, the process of assessing pathological conditions from images may be achieved linearly, by first acquiring the image through the camera, performing quality control on the image, and depending on the resulting quality check, passing it to the disease classification module, which would output a final result, or pass a value (such as, via a message) back to the camera application module 401 to re-take the image.

In an initial step, the camera application module 401 can obtain images of the retina. The images can be obtained from video data captured by the camera. The camera application module 401 can then send a notification to the CCC 402. The CCC 402 can store the received images in a database and pass on the notification to the image quality assessment module (Module X, 4031), indicating that there is a new image to be assessed for good or poor quality. This notification can be a specific message containing the path (physical memory location of where the image is stored) of the image and any other relevant information (such as, which eye the image has been acquired from, which field of view, or the like). Upon receipt of this notification, the image quality assessment module (Module X, 4031) can assess the image and output a quality indication (such as, a binary value) indicating whether the image has passed the quality check. The resulting output can be compiled and sent to the CCC 402. In some implementations, the image quality check can be done manually, for instance by displaying the acquired image on the display 130. A prompt can then ask the user to confirm whether the image is good or poor quality, and user's response can be sent to the CCC 402. The user could also select a region of interest via the display 130 in order to select a portion of the image that the user deem to be of good image quality to be passed to further modules.

Depending on the message received, the CCC 402 can have multiple choices, such as whether to retake the image or move on to the next image. Assuming that the image has passed the quality check, the CCC 402 can choose to perform several actions. For example, if there are no memory allocation issues (for instance, if there is sufficient memory space), the CCC 402 can choose to send a “Good quality image” (or similar) message to the disease classification module 4032, while notifying the camera application module 401 to acquire the next image. These tasks can be performed in parallel. Alternatively, if the CCC 402 determines that performing these tasks in parallel would result in memory allocation errors, it can decide to inform the camera application module 401 to acquire a new image. After the new image has been acquired, the CCC 402 can proceed to checking the quality of that image before sending it (along with at least some or all previously acquired and quality checked images) to the disease classification module 4032. Depending on the other connected modules, the CCC 402 can have the ability to prioritize tasks and as such free up memory allocation in order to allow for the parallel processing of the image acquisition and disease classification.

Subsequently, the CCC 402 can send a notification to the disease classification module 4032 (possibly along with other information) to allow the disease classification module to run and produce a final output of the disease severity score. The disease classification module 4032 can include several components that generate features from the image and aggregate the features to produce a disease severity score. The disease severity score can be one or more of a discrete set of outputs indicating severity of disease (such as, 0 no disease, 1 mild disease, 2 moderate disease, etc.), a binary output indicating the presence or absence of disease, or a probabilistic output indicating the likelihood of disease. The probabilistic output can be obtained by model calibration or using a machine learning model with uncertainty (for example, one or more Bayesian neural networks). The final output can be sent back to the CCC 402 that, depending on the resulting message information, can choose to send a message to another module tasked with displaying the final output of the disease severity score to the user. The resulting message information can cause the CCC 402 to store the disease severity score per image and the patient's overall disease severity score in the database. A second message can cause the CCC 402 to provide the patient's disease severity score to the user, such as display on the display 130.

The communication between the CCC 402 and various modules can occur through channels 41, 42, 47, and 48. As described herein, a channel can be a relay pathway between modules. Each individual module can join (or subscribe) and read/write (or publish) to the relevant channels as needed. Each channel can have a specific key-value pair message that is tailored to the relevant communication. For example, the camera application module 401 will subscribe to the “results_channel” 47. The image quality assessment module 4031 will subscribe to “quality_channel” 41. The disease classification module 4032 will subscribe to the “ml_channel” 42. The “quality_channel” 41 can be used between the camera application module 401, CCC 402, and the image quality assessment module 4031. This channel may utilize one or more of the following keys: “eye_id” (string), “image_path” (string) and “result” (string or binary). In another example, there may be multiple channels of each type, such as a “quality_results_channel” 47 and a “disease_detection_results_channel” 48, and the modules may subscribe to one or more of the relevant channels. The camera application module 401 may subscribe to one or both the “quality_results_channel” 47 and the “disease_detection_results_channel” 48.

By using the channels, only relevant information can be passed, thereby leading to a more streamlined and efficient implementation. Because the key-value pairs can be defined independently for each channel, the CCC 402 can create an efficient communication system by utilizing a database of channel subscriptions to lookup the status of various modules (for instance, using the watchdog channels, as described herein). For instance, the CCC 402 can determine the relationship between the various modules by identifying which modules are subscribed to which channel. Communications between various modules can be centralized through the CCC 402. For example, as the image quality assessment module 4031 and the disease classification module 4032 do not subscribe to each other's channel, there is no communication protocol that can take place directly between these modules.

In addition to or instead of the module specific channels, there may be system specific channels to ensure seamless communication. These additional channels can inform on the status of the individual modules and ensure that the at least some or all individuals modules are ready to be executed. These channels may be referred to as watchdog channels, over which messages (which can be short) can be relayed to the individual modules to ensure their availability. The messages can be transmitted periodically (such as, every 30 seconds or less or more) over the watchdog channels. As such, there can be a lot of flexibility in setting up communication pathways to efficiently run individual modules and ensure the instrument as a whole is operating efficiently and successfully.

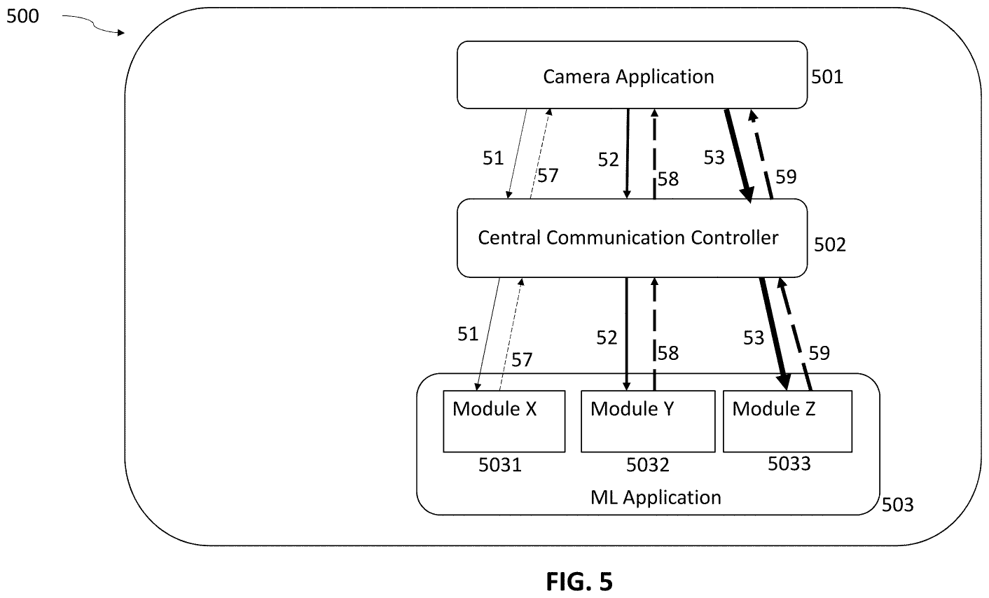

FIG. 5 schematically illustrates a modular architecture 500 for a retinal diagnostics instrument (which can be a closed loop system). The architecture 500 can implement a dual-purpose classification modular AI image analysis process. Any of the modules illustrated in FIG. 5 can be software or firmware modules stored in a memory. Similar to FIG. 4, the architecture 500 can include a camera application module 501, a master controller or central communication controller (CCC) 502, and a machine learning (ML) application module 503.

For example, the dual-purpose classification modular AI image analysis process can perform both diabetic retinopathy classification and glaucoma classification. The ML application module 503 can include three separate modules: an image quality assessment module (Module X, 5031), a diabetic retinopathy classification module (Module Y, 5032), and a glaucoma classification module (Module Z, 5033).

The image quality assessment module 5031 may be a non-linear classifier, such as a support vector machine (SVM), and can check for different image features (such as, poor brightness, poor image saturation, out of focus or blurry, or the like) before assigning a quality indication (such as, a binary value). The image quality assessment module 5031 can takes as input one image and outputs the quality indication. The CCC 502 can send images to be analyzed to the image quality assessment module 5031.

The diabetic retinopathy classification module 5032 can be a convolutional neural network that takes in one or many images and outputs a diabetic retinopathy disease severity score. The CCC 502 can provide the images to be analyzed to the diabetic retinopathy classification module 5032.

The glaucoma classification module 5033 can be another neural network which makes use of the same retina images as the diabetic retinopathy classification module 5031. If the CCC 502 determines that the system is able to perform any analysis in parallel, it can send the images to both the diabetic retinopathy classification module 5032 and the glaucoma classification module 5033 at the same time.

The communication between the CCC 502 and various modules can occur through channels 51, 52, 53, 57, 58, and 59 (which can be similar to the channels described above in connection with FIG. 4, with the channel 53 being a new channel). Each of the channels can have a specific key-value pair messages that are tailored to the relevant communication. For example, the camera application module 501 will join the “results_channel” 57. The image quality assessment module 5031 will subscribe to the channel 51, the diabetic retinopathy classification module 5032 will subscribe to the channel 52, and the glaucoma classification module 5033 will subscribe to channel 53. In another example, there may be multiple channels of each type, such as a “quality_results_channel” 57, a “diabetic retinopathy_detection_results_channel” 58, and a “glaucoma_detection_results_channel” 59, and the modules may subscribe to one or more of the relevant channels. The camera application module 501 may subscribe to some or all of the “quality_results_channel” 57, the “diabetic retinopathy_detection_results_channel” 58, and the “glaucoma_detection_results_channel” 59.

An upgrade from the modular architecture 400 shown in FIG. 4 to the architecture 500 shown in FIG. 5 can performed as follows. There can be an update of the instrument (and/or the CCC) with one or more new communication channels defining the appropriate key-value pairs and an addition of the glaucoma classification module 5033. The illustrated and disclosed architecture can allow for easy modification to the system, without affecting the existing modules or channels.

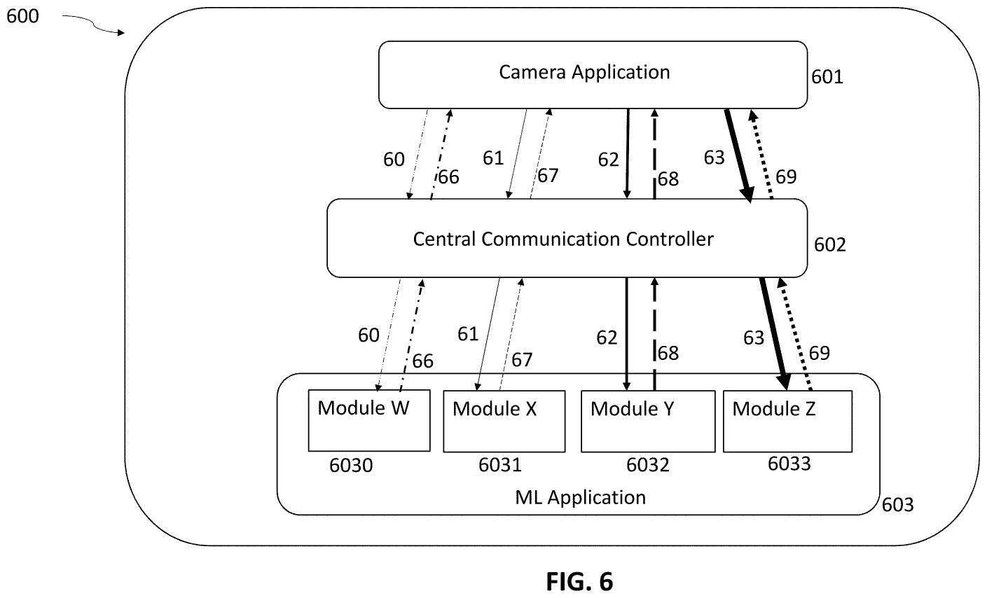

FIG. 6 schematically illustrates a modular architecture 600 for a retinal diagnostics instrument (which can be a closed loop system). The architecture 600 can implement a dual-purpose classification modular AI image analysis process. Any of the modules illustrated in FIG. 6 can be software or firmware modules stored in a memory. Similar to FIGS. 4 and 5, the architecture 600 can include a camera application module 601, a master controller or central communication controller (CCC) 602, and a machine learning (ML) application module 603.

For example, the modular AI image analysis can perform diabetic retinopathy classification with several intermediate processes. The ML application module 603 can include four separate modules: an image quality assessment module (Module W, 6030), a feature extraction module (Module X, 6031), a microaneurysm extraction module (Module Y, 6032) that identifies the possible location of microaneurysms in the retinal image, and a diabetic retinopathy classification module (Module Z, 6033). Each of these modules can be a machine learning model, such as a nonlinear classifier or a CNN.

The captured images can be provided to the image quality assessment module 6030. Depending on the resulting output from that module and the memory resources, the images may be provided to both the feature extraction module 6031 and microaneurysm extraction module 6032. The resulting output of the feature extraction module 6031 and microaneurysm extraction module 6032 can be stored in the database and provided to the diabetic retinopathy classification module 6033 by the CCC 602.

In some implementations, the diabetic retinopathy classification module 6033 may rely on the feature extraction module 6031 to extract image-based features beforehand that will be used as additional input information to the diabetic retinopathy classification module 6033. The image-based features may be retina disk identification (for example, determined via the delineation of blood vessels). Having the feature extraction module 6031 separate from the diabetic retinopathy classification module 6033 can provide several advantages, including the ability to store intermediate results and to easily upgrade the module without affecting the classification (such as, the classification neural network). The intermediate results can be useful for one or more disease classification modules.

The CCC 602 can facilitate transfer of the relevant information between modules. As it can perform status checks on the various modules (which can be done continuously), the CCC 602 can pass the data at the appropriate times and in the correct order, all while understanding the needs of the system as a whole (for example, with respect to the memory allocation). The communication between the CCC 602 and various modules can occur through channels 60, 61, 62, 63, 66, 67, 68, and 69 (which can be similar to the channels described above in connection with FIGS. 4 and 5). Each channel can have a specific key-value pair message that is tailored to the relevant communication.

For example, the camera application module 601 will join the “results_channel” 67. The image quality assessment module 6030 will subscribe to channel 60, the feature extraction module 6031 will subscribe to channel 61, the microaneurysm extraction module 6032 will subscribe to channel 62, and the diabetic retinopathy classification module 6033 will subscribe to channel 63. In another example, there may be multiple channels of each type, such as a “quality_results_channel” 66, a “feature extraction_results_channel” 67, a “microaneurysm extraction_results_channel” 68 and a “diabetic retinopathy_detection_results_channel” 69, and the modules may subscribe to one or more of the relevant channels. The camera application module 601 may subscribe to some or all of the “quality_results_channel” 66, “feature extraction_results_channel” 67, “microaneurysm extraction_results_channel” 68 and “diabetic retinopathy_detection_results_channel” 69.

Understanding the status of the system as a whole allows the CCC 602 to either pass in real-time, parallel, or sequentially the data serving as the input to the various modules. For instance, the CCC 602 could choose to forward the images having passed the image quality assessment check at image quality assessment module 6030 to both the feature extraction module 6031 and microaneurysm extraction module 6032 independently or simultaneously, to be analyzed in parallel, or chose which module to forward the image to, depending on the memory allocation.

An upgrade of the architecture 500 shown in FIG. 5 to the architecture 600 shown in FIG. 6 can be performed as described above.

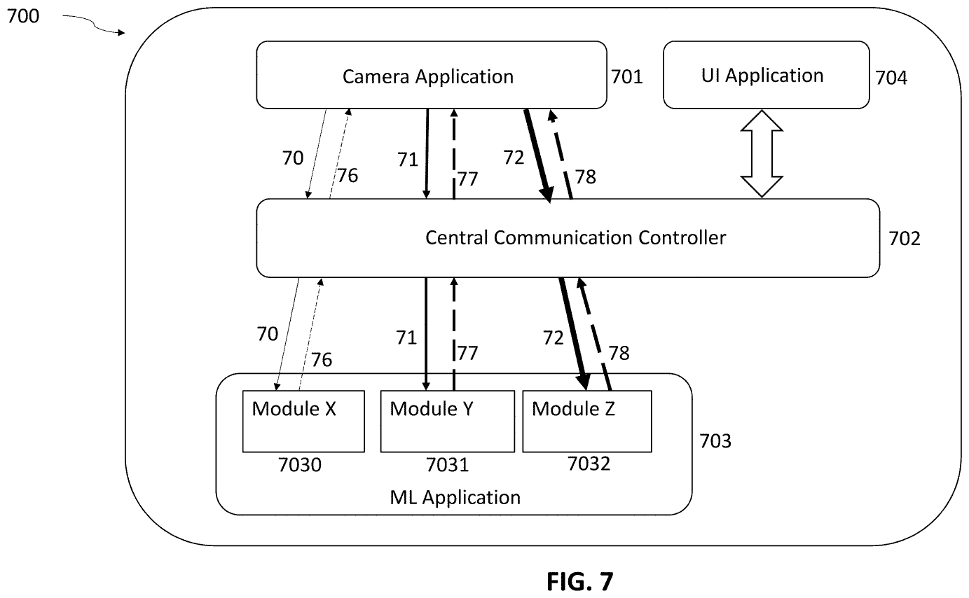

FIG. 7 schematically illustrates a modular architecture 700 for a retinal diagnostics instrument that relies on user input. The architecture 700 can implement a dual-purpose classification modular AI image analysis process as described in FIG. 5 and FIG. 6 or implement the process described in connection with FIG. 3. Any of the modules illustrated in FIG. 7 can be software or firmware modules stored in a memory. Similar to FIGS. 4, 5, and 6, the architecture 700 can include a camera application module 701, a master controller or central communication controller (CCC) 702, a machine learning (ML) application module 703. The architecture 700 can also include a user interface module 704 (and, in some cases, an electronic medical record module which may be included in addition to or in place of the user interface module 704). The user interface module 704 can receive one or more inputs from the user via the user interface (such as, via the display 130). For example, the user can input patient information which can be fed into one or more machine learning models. As another example, the user can manually rate the quality of an image and override any automatic image quality assessment modules.

For instance, the modular AI image analysis can perform diabetic retinopathy classification with several intermediate processes. The ML application module 703 can include three separate modules: an image correction module (Module X, 7030), a feature extraction module (Module Y, 7031), and a diabetic retinopathy classification module (Module Z, 7032). Each of these modules can be a machine learning model (for example, based on a CNN). Module 7030 and module 7031 can be based on nonlinear feature extractors, for example, based on machine learning models or based on a series of filter banks.

The image correction module 7030 can improve the quality of an acquired image (for example, by image denoising, removing image glare, deblurring and sharpening, and improving focus on the retina). In some implementations, module 7030 may be implemented as a series of Fourier-based or Wavelet-based feature banks (for example based on learned steerable filters) or may be the output of a CNN-based image correction model. The image correction module 7030 can take as input one image and output a corrected image. The CCC 702 can send images to be corrected to the image correction module 7030.

The user interface module 704 can communicate with the user via the user interface (such as, one or more of a touch screen or physical buttons) to allow for user input. For example, the user interface module 704 may request that the user confirm the quality of an acquired image or provide patient information (such as, patient age, weight, and gender). The user interface module 704 can receive a notification from the CCC 702 when user input is needed. For example, an image acquired by the user can be communicated to the CCC 702 and be passed to the image correction module 7030. The image correction module 7030 can pass a corrected image to the CCC 702, which is relayed to user interface module 704. The user interface module 704 can display the corrected image on the screen. Subsequently, the use interface module 704 can request that the user confirm if the image is of sufficient quality or to retake the image. If the image is of sufficient quality, the CCC 702 can one or more of either prompt the user to acquire the next image or pass the corrected image to the modules 7031 and 7032 to output a diagnosis for diabetic retinopathy. The user interface module 704 could be used to have the user select a region of interest in the image as part of the manual image correction. The selected region of interest would be passed to the CCC 702, which in turn would pass it to the modules 7031 and 7032.

The CCC 702 can facilitate transfer of the relevant information between the modules. As it can perform status checks on the various modules (which can be done continuously), the CCC 702 can pass the data at the appropriate times and in the correct order, all while understanding the needs of the system as a whole (for example, with respect to the memory allocation). The communication between the CCC 702 and various modules can occur through channels 70, 71, 72, 76, 77, and 78 (which can be similar to the channels described above in connection with FIGS. 4, 5, and 6, and particularly FIG. 6). Each channel can have a specific key-value pair message that is tailored to the relevant communication.

For example, the camera application module 701 will join the “results_channel” 77. The image quality correction module 7030 will subscribe to channel 70, the feature extraction module 7031 will subscribe to channel 71, and the diabetic retinopathy classification module 7033 will subscribe to channel 72. The user interface module 704 will subscribe to one or more of channels 70, 76, 77, and 78. Subscription to channel 76 may be for accepting or overriding the image quality result. Subscription to channel 72 may be for inputting patient information utilized for diabetic retinopathy classification performed by module 7032 (such as, patient weight). Subscription to channel 78 may be to accept or override diabetic retinopathy classification output by module 7032. Subscription to channel 77 may be for displaying the extracted features and confirming the presence of features (such as, presence of an optical disk that the module 7031 has extracted from the image).

There may be multiple channels of each type, such as an “image_correction_channel” 70 a “feature_extraction_results_channel” 77 and a “diabetic_retinopathy_detection_results_channnel” 78, and the modules may subscribe to one or more of the relevant channels. The camera application module 701 may subscribe to some or all of the “image_correction_channel” 70, “quality_results_channel” 76, “feature extraction_results_channel” 77, and “diabetic retinopathy_detection_results_channel” 78.

Understanding the status of the system as a whole allows the CCC 702 to either pass in real-time, parallel, or sequentially the data serving as the input to the various modules. For instance, the CCC 702 could choose to forward the images having been quality corrected at the module 7030 to the user for verification assessment (via the user interface module 704), then the results to the feature extraction module 7031 for analysis or to the camera application module 703 to acquire the next image (or both in parallel).

An upgrade of the architecture 600 shown in FIG. 6 to the architecture 700 shown in FIG. 7 can be performed as described above.

### Additional Examples

Example 1: A retinal diagnostics instrument comprising:
a housing;
an imaging device supported by the housing, the imaging device configured to capture image data of an eye of a patient, the image data comprising one or more still images or video data; and
an electronic processing circuitry supported by the housing, the electronic processing circuitry comprising a memory and one or more processors, the memory storing executable instructions that, when executed by the one or more processors, cause the one or more processors to execute a communication control method comprising:
   providing input data to and receiving output data from a plurality of modules stored in the memory, the plurality of modules configured to facilitate diagnosing a retina of the eye of the patient using the image data; and
   causing independent processing of the input data by any first module of the plurality of modules and any second module of the plurality of modules, any second module being different than any first module, wherein processing of the input data by any first module does not depend on directly providing data to or receiving data from any second module and vice versa.
      Example 2: The instrument of any of the preceding examples, wherein the communication control method further comprises providing the input data to and receiving the output data from the plurality of modules over a plurality of channels, and wherein a module of the plurality of modules is configured to join a channel of the plurality of channels to receive the input data or provide the output data.
      Example 3: The instrument of example 2, wherein communication of data over the plurality of channels is performed using one or more key and value pairs.
      Example 4: The instrument of example 3, wherein the one or more key and value pairs are specific to each channel of the plurality of channels.
      Example 5: The instrument of any of the preceding examples, wherein the communication control method further comprises determining status of the plurality of modules by transmitting requests and receiving status information from the plurality of modules over a plurality of watchdog channels.
      Example 6: The instrument of any of the preceding examples, wherein the executable instructions further cause deactivating or upgrading any module of the plurality of modules or adding one or more new modules, and wherein the deactivating or upgrading or adding does not affect operation of any of the other modules of the plurality of modules.
      Example 7: The instrument of any of the preceding examples, wherein at least one of any first module or any second module comprises a machine learning module configured to identify one or more diseases of the retina.
      Example 8: The instrument of any of the preceding examples, further comprising a user interface, wherein the communication control method further comprises:
by a third module of the plurality of modules, receiving user input provided via the user interface and communicating the user input to at least one of any first module or any second module.
   Example 9: The instrument of example 8, wherein the user input comprises one or more of patient information, selection of a portion of the image data, or quality assessment of the image data.
   Example 10: The instrument of any of the preceding examples, wherein the imaging device comprises a camera.
   Example 11: The instrument of any of the preceding examples, further comprising a cup positioned at a distal end of the housing, the cup configured to be an interface between instrument and the eye of the patient.
   Example 12: The instrument of example 11, wherein the cup is disposable.
   Example 13: The instrument of any of the preceding examples, wherein the housing is portable.
   Example 14: The instrument of any of the preceding examples, wherein the housing comprises a body and a handle, the handle connected to the body and configured to be held by a user.
   Example 15: The instrument of any of the preceding examples, wherein the image data comprises at least one still image obtained from the video data.
   Example 16: A method of operating a retinal diagnostics instrument, the method comprising:
by electronic processing circuitry of the retinal diagnostics instrument:
   providing input data to and receiving output data from a plurality of modules stored in a memory of the retinal diagnostics instrument, the plurality of modules configured to facilitate diagnosing a retina of an eye of a patient using an image data of the eye of the patient; and
   causing independent processing of the input data by any first module of the plurality of modules and any second module of the plurality of modules, any second module being different than any first module, wherein processing of the input data by any first module does not depend on directly providing data to or receiving data from any second module and vice versa.
      Example 17: The method of example 16, further comprising providing the input data to and receiving the output data from the plurality of modules over a plurality of channels, wherein a module of the plurality of modules is configured to join a channel of the plurality of channels to receive the input data or provide the output data.
      Example 18: The method of example 17, wherein communication of data over the plurality of channels is performed using one or more key and value pairs.
      Example 19: The method of example 18, wherein the one or more key and value pairs are specific to each channel of the plurality of channels.
      Example 20: The method of any of examples 16 to 19, further comprising determining status of the plurality of modules by transmitting requests and receiving status information from the plurality of modules over a plurality of watchdog channels.
      Example 21: The method of any of examples 16 to 20, further comprising deactivating or upgrading any module of the plurality of modules or adding one or more new modules, and wherein the deactivating or upgrading or adding does not affect operation of any of the other modules of the plurality of modules.
      Example 22: The method of any of examples 16 to 21, wherein at least one of any first module or any second module comprises a machine learning module configured to identify one or more diseases of the retina.
      Example 23: The method of any of examples 16 to 22, wherein the image data comprises at least one still image obtained from video data.
      Example 24: The method of any of examples 16 to 23, further comprising:
by a third module of the plurality of modules, receiving user input provided via a user interface of the retinal diagnostics instrument and communicating the user input to at least one of any first module or any second module.
   Example 25: The method of example 24, wherein the user input comprises one or more of patient information, selection of a portion of the image data, or quality assessment of the image data.

### Other Variations

Although the foregoing provides one or more examples of live image or video analysis on a retina camera, disclosed systems, devices, and methods are not limited to retina cameras, but can be extended to any diagnostics device, such as an otoscope, dermatology scope, or the like. Although the foregoing provides one or more examples of a portable medical diagnostics device, the approaches disclosed herein can be utilized by non-portable (such as, table top) diagnostics devices.

Although the foregoing provides one or more examples of live image or video analysis on-board, disclosed systems, devices, and methods are not so limited and can be utilized by cloud-based systems, particularly in situations where reliable network connectivity is available.

Example implementations are described with reference to classification of the eye tissue, but the techniques may also be applied to the classification of other tissue types. More specifically, the approach of visualizing the effects of multiple different tissue segmentations as an aid for the user to understand their effects, and hence to gain insight into the underlying explanation for the output classification, is generally applicable to many different tissue regions and types. For example, X-ray, ultrasound or MM images all produce 2D or 3D images of regions of the body, and it will be apparent that the image segmentation neural network described may be used to segment different tissue types from such images. The segmented region may then be analyzed by the classification neural network to classify the image data, for example identify one or more pathologies and/or determine one or more clinical referral decisions. Other implementations of the system may be used for screening for other pathologies in other body regions.

Any of the transmission of data described herein can be performed securely. For example, one or more of encryption, https protocol, secure VPN connection, error checking, confirmation of delivery, or the like can be utilized.

The design may vary as components may be added, removed, or modified. Depending on the embodiment, certain acts, events, or functions of any of the processes or algorithms described herein can be performed in a different sequence, can be added, merged, or left out altogether (e.g., not all described operations or events are necessary for the practice of the algorithm). Moreover, in certain embodiments, operations or events can be performed concurrently, e.g., through multi-threaded processing, interrupt processing, or multiple processors or processor cores or on other parallel architectures, rather than sequentially.

The various illustrative logical blocks, modules, routines, and algorithm steps described in connection with the embodiments disclosed herein can be implemented as electronic hardware, or combinations of electronic hardware and computer software. To clearly illustrate this interchangeability of hardware and software, various illustrative components, blocks, modules, and steps have been described above generally in terms of their functionality. Whether such functionality is implemented as hardware, or as software that runs on hardware, depends upon the particular application and design constraints imposed on the overall system. The described functionality can be implemented in varying ways for each particular application, but such implementation decisions should not be interpreted as causing a departure from the scope of the disclosure.

Moreover, the various illustrative logical blocks and modules described in connection with the embodiments disclosed herein can be implemented or performed by a machine, such as a general purpose processor device, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor device can be a microprocessor, but in the alternative, the processor device can be a controller, microcontroller, or state machine, combinations of the same, or the like. A processor device can include electronic circuitry configured to process computer-executable instructions. In another embodiment, a processor device includes an FPGA or other programmable device that performs logic operations without processing computer-executable instructions. A processor device can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the embodiments disclosed herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An example storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without other input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y, Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, or within less than 0.01% of the stated amount.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations.

While the above detailed description has shown, described, and pointed out novel features as applied to various embodiments, it can be understood that various omissions, substitutions, and changes in the form and details of the devices or algorithms illustrated can be made without departing from the spirit of the disclosure. As can be recognized, certain embodiments described herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

What is claimed is:

1. A retinal diagnostics instrument comprising:
a housing;
an imaging device supported by the housing, the imaging device configured to capture image data of an eye of a patient, the image data comprising one or more still images or video data; and
an electronic processing circuitry supported by the housing, the electronic processing circuitry comprising a memory and one or more processors, the memory storing executable instructions that, when executed by the one or more processors, cause the one or more processors to execute a communication control method comprising:
providing input data to and receiving output data from a plurality of modules stored in the memory, the plurality of modules configured to facilitate diagnosing a retina of the eye of the patient using the image data; and
causing independent processing of the input data by any first module of the plurality of modules and any second module of the plurality of modules, any second module being different than any first module, wherein processing of the input data by any first module does not depend on directly providing data to or receiving data from any second module and vice versa.

2. The instrument of claim 1, wherein the communication control method further comprises providing the input data to and receiving the output data from the plurality of modules over a plurality of channels, and wherein a module of the plurality of modules is configured to join a channel of the plurality of channels to receive the input data or provide the output data.

3. The instrument of claim 2, wherein communication of data over the plurality of channels is performed using one or more key and value pairs.

4. The instrument of claim 3, wherein the one or more key and value pairs are specific to each channel of the plurality of channels.

5. The instrument of claim 1, wherein the communication control method further comprises determining status of the plurality of modules by transmitting requests and receiving status information from the plurality of modules over a plurality of watchdog channels.

6. The instrument of claim 1, wherein the executable instructions further cause deactivating or upgrading any module of the plurality of modules or adding one or more new modules, and wherein the deactivating or upgrading or adding does not affect operation of any of the other modules of the plurality of modules.

7. The instrument of claim 1, wherein at least one of any first module or any second module comprises a machine learning module configured to identify one or more diseases of the retina.

8. The instrument of claim 1, further comprising a user interface, wherein the communication control method further comprises:
by a third module of the plurality of modules, receiving user input provided via the user interface and communicating the user input to at least one of any first module or any second module.

9. The instrument of claim 8, wherein the user input comprises one or more of patient information, selection of a portion of the image data, or quality assessment of the image data.

10. The instrument of claim 1, wherein the imaging device comprises a camera.

11. The instrument of claim 1, wherein the housing is portable, and wherein the housing comprises a body and a handle, the handle connected to the body and configured to be held by a user.

12. The instrument of claim 1, wherein the image data comprises at least one still image obtained from the video data.

13. A method of operating a retinal diagnostics instrument, the method comprising:
by electronic processing circuitry of the retinal diagnostics instrument:
providing input data to and receiving output data from a plurality of modules stored in a memory of the retinal diagnostics instrument, the plurality of modules configured to facilitate diagnosing a retina of an eye of a patient using an image data of the eye of the patient; and
causing independent processing of the input data by any first module of the plurality of modules and any second module of the plurality of modules, any second module being different than any first module, wherein processing of the input data by any first module does not depend on directly providing data to or receiving data from any second module and vice versa.

14. The method of claim 13, further comprising providing the input data to and receiving the output data from the plurality of modules over a plurality of channels, wherein a module of the plurality of modules is configured to join a channel of the plurality of channels to receive the input data or provide the output data.

15. The method of claim 14, wherein communication of data over the plurality of channels is performed using one or more key and value pairs.

16. The method of claim 15, wherein the one or more key and value pairs are specific to each channel of the plurality of channels.

17. The method of claim 13, further comprising determining status of the plurality of modules by transmitting requests and receiving status information from the plurality of modules over a plurality of watchdog channels.

18. The method of claim 13, further comprising deactivating or upgrading any module of the plurality of modules or adding one or more new modules, and wherein the deactivating or upgrading or adding does not affect operation of any of the other modules of the plurality of modules.

19. The method of claim 13, wherein at least one of any first module or any second module comprises a machine learning module configured to identify one or more diseases of the retina.

20. The method of claim 13, further comprising:
by a third module of the plurality of modules, receiving user input provided via a user interface of the retinal diagnostics instrument and communicating the user input to at least one of any first module or any second module.

21. The method of claim 20, wherein the user input comprises one or more of patient information, selection of a portion of the image data, or quality assessment of the image data.
